(19) United States
(12) Patent Application Publication
(10) Pub. No.: US 2015/0352228 A1
(43) Pub. Date: Dec. 10, 2015

(54) METHODS AND COMPOSITIONS FOR ENHANCING TRANSDUCTION EFFICIENCY OF RETROVIRAL VECTORS

(71) Applicants: Bruce TORBET, Encinitas, CA (US); Cathy WANG, La Jolla, CA (US); THE SCRIPPS RESEARCH INSTITUTE, La Jolla, CA (US)

(72) Inventors: Bruce Torbett, Encinitas, CA (US); Cathy X. Wang, La Jolla, CA (US)

(21) Appl. No.: 14/760,271

(22) PCT Filed: May 17, 2013
(86) PCT No.: PCT/US2013/000136

§ 371 Date: Jul. 10, 2015

Related U.S. Application Data
(60) Provisional application No. 61/751,374, filed on Jan. 11, 2013.

(51) Int. Cl. A61K 48/00; A61K 31/436; C12N 5/0789

(52) U.S. Cl. CPC A61K 48/0008; C12N 5/0647; A61K 31/436; C12N 2501/145; C12N 2501/22; C12N 2501/2306; C12N 2501/26; C12N 2501/125; C12N 2501/04

(57) ABSTRACT

The present invention provides methods for enhancing transduction efficiency of a viral vector into a host cell such as a stem cell. The methods involve transducing the host cell with the vector in the presence of an inhibitor of mTOR complexes (e.g., rapamycin or analog compound thereof). Also provided in the invention are kits or pharmaceutical combinations for delivering a therapeutic agent into a target cell with enhanced targeting frequency and payload delivery. The kits or pharmaceutical combinations typically contain a viral vector encoding the therapeutic agent, and an inhibitor of mTOR complexes.

### CROSS-REFERENCE TO RELATED APPLICATIONS

The subject patent application claims the benefit of priority to U.S. Provisional Patent Application No. 61/751,374 (filed Jan. 11, 2013). The full disclosure of the priority application is incorporated herein by reference in its entirety and for all purposes.

### COPYRIGHT NOTIFICATION

Pursuant to 37 C.F.R. §1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### BACKGROUND OF THE INVENTION

Viruses are highly efficient at nucleic acid delivery to specific cell types, while often avoiding detection by the infected host immune system. These features make certain viruses attractive candidates as gene-delivery vehicles for use in gene therapies. Retroviral vectors are the most commonly used gene delivery vehicles. The retroviral genome becomes integrated into host chromosomal DNA, ensuring its long-term persistence and stable transmission to all future progeny of the transduced cell and making retroviral vector suitable for permanent genetic modification. Retroviral based vectors can be manufactured in large quantities, which allow their standardization and use in pharmaceutical preparations.

Hematopoietic stem cells (HSCs), long-lived precursors to the entire hematopoietic system, are intrinsically refractory to HIV-1 replication. Human CD34⁺ hematopoietic stem and progenitor cells can be infected in vitro at low levels, but occurrence of in vivo infection remains controversial. Similarly, they are refractory to transduction by HIV-1 based lentiviral vectors, greatly hampering the efficacy of HSC gene therapy. NOD/SCID-repopulating cells experimentally defined as truly primitive HSCs show only low levels of lentiviral-mediated gene marking, which cannot be overcome even by extremely high vector-to-cell ratios. The block is thought to occur post-entry, as primary HSCs express HIV-1 receptors, and lentiviral vectors are commonly pseudotyped with the vesicular stomatitis virus glycoprotein (VSV-G) to allow for ubiquitous tropism.

There is a need in the art for means for more efficiently transducing retroviral vectors, esp. lentiviral vector such as HIV based vectors, into host cells (e.g., stem cells) in gene transfer. The present invention addresses this and other needs.

### SUMMARY OF THE INVENTION

In one aspect, the invention provides methods for enhancing transduction efficiency of a viral vector into a stem cell. The methods entail transducing the stem cell with the vector in the presence of a compound that inhibits mTOR complexes. In some of the methods, the employed inhibitor compound is an mTOR inhibitor which targets the mTOR kinase. In some methods, the employed mTOR inhibitor is rapamycin or an analog compound of rapamycin. Some other methods employ an ATP-competitive mTOR inhibitor, e.g., Torin 1. Some of the methods are directed to enhancing transduction efficiency of recombinant retroviral vectors, adenoviral vectors or adeno-associated viral vectors. In some methods, the employed viral vector is a lentiviral vector. In some methods, the viral vector is a HIV-1 based vector.

Some methods of the invention are directed to enhancing transduction efficiency of a viral vector into a hematopoietic stem cell (HSC), an embryonic stem cell or a mesenchymal stem cell. Preferably, the employed stem cell is a hematopoietic stem cell. The stem cell suitable for the invention can be isolated from various sources or biological samples, e.g., peripheral blood, umbilical cord blood or bone marrow. In some preferred embodiments, the employed stem cell is human CD34⁺ cell.

In some methods of the invention, the stem cell can be optionally pre-stimulated with at least one cytokine prior to transduction of the vector. For example, the stem cell can be pre-stimulated with TPO, CSF, IL-6, Flt-3 or SCF. In some methods of the invention, the viral vector is transduced into the stem cell at a multiplicity of infection (MOI) of 5, 10, 25, 50, 100 or higher. In some methods, the inhibitor of mTOR complexes (e.g., rapamycin) is present during the entire transduction process or at specific intervals. In some methods, the viral vector can encode a therapeutic agent. In some methods, the employed viral vector is a non-integrating lentiviral vector.

In another aspect, the invention provides kits or pharmaceutical combinations for delivering a therapeutic agent into a target cell with enhanced targeting frequency and payload delivery. The kits typically contain (a) a viral vector encoding the therapeutic agent, and (b) an inhibitor of mTOR complexes. In some kits of the invention, the inhibitor of mTOR complexes is a compound that targets the mTOR kinase (mTOR inhibitor). In some kits, the mTOR inhibitor is rapamycin or an analog compound of rapamycin. In some other kits, an ATP-competitive inhibitor of mTOR is provided (e.g., Torin 1). Some of the kits are specifically intended for delivering a therapeutic agent to hematopoietic stem cells (HSCs). In some of the kits, the employed viral vector is a lentiviral vector. Some of the kits of the invention are designed for delivering a therapeutic agent that is a polynucleotide agent or a polypeptide agent. The kits of the invention can optionally further contain a target cell into which the therapeutic agent is to be delivered. In some of the kits, the target cell for delivering a therapeutic agent is human CD34⁺ cell.

A further understanding of the nature and advantages of the present invention may be realized by reference to the remaining portions of the specification and claims.

### DESCRIPTION OF THE DRAWINGS

FIGS. 1A-1D show that rapamycin increases lentiviral transduction efficiency in human CD34⁺ cells. CD34⁺ cells from either (A) cord blood or (B) adult bone marrow were transduced with HIV-1 based lentiviral vectors at an MOI=50, in the presence of indicated concentrations of rapamycin. Cells were transduced either directly after isolation, or after 24 hours of pre-stimulation in a cytokine cocktail. Transduction of cord blood CD34⁺ cells, either (C) non-stimulated or (D) stimulated, were further tested at a range of MOIs, at the indicated concentrations of rapamycin. Percentages of cells expressing GFP were analyzed by flow cytometry 11-14 days after transduction. Line represents mean of duplicate transductions.

FIGS. 2A-2H show that CD34⁺ cells transduced in the presence of rapamycin maintain long-term and serial repopulating potential in NSG mice. Stimulated cord blood CD34⁺ cells were transduced with MOI=25 in the presence of indicated concentrations of rapamycin. Portions of transduced cells were used in liquid culture and CFU assay; the rest were injected into irradiated NSG mice. (A) The colony forming efficiencies (p=0.0907 for 0 μg/ml vs 20 μg/ml), (B) colony types, and (C) percentages of GFP⁺ colonies or cells were analyzed by fluorescence microscopy or flow cytometry as previously stated. Error bars represent standard deviations of triplicate cultures. (D) NSG mice were sacrificed 19 weeks post injection. Reconstitution levels in the bone marrow (p=0.2154 for 0 μg/ml vs 20 μg/ml), (E) percentages of GFP⁺ cells in each human hematopoietic lineage (p=0.0018 and 0.0005 for 0 μg/ml vs 10 μg/ml and 20 μg/ml respectively), and (F) mean fluorescent intensity in human CD45⁺ cells (p=0.0256 and 0.1324 for 0 μg/ml vs 10 μg/ml and 20 μg/ml respectively) were analyzed by flow cytometry. (G) Proviral copy numbers in mouse bone marrow cells were quantified by qPCR and adjusted for reconstitution levels to reflect integration in human CD45⁺ cells (p=0.0619 and 0.0640 for 0 μg/ml vs 10 μg/ml and 20 μg/ml respectively). (H) To examine serial engraftment potential, cord blood CD34⁺ cells transduced with MOI=50 in the presence of 10 ng/ml rapamycin were injected into 1′ recipient NSG mice, the bone marrow of which following reconstitution were injected into 2′ recipient NSG mice. Both sets of bone marrow were harvested and analyzed 12 weeks post injection for GFP expression by flow cytometry. Line represents mean of each mouse group.

FIG. 3 shows that rapamycin increases transduction efficiency of integrase-defective lentiviral vectors (IDLVs). Human cord blood CD34⁺ cells were stimulated for 24 h and transduced with IDLVs, MOI=50, with or without 10 ng/μl rapamycin. GFP expression was assayed by flow cytometry every three days from 2-14 days post transduction. Data represent mean and standard deviation of independent transductions of two separate donors. 2 dpt, p<0.0001; 5 dpt, p=0.03 by Student t test.

FIGS. 4A-4F show that rapamycin increases transduction efficiency of wild type and integrase-defective lentiviral vectors in mouse Lin-cells. Mouse Lin-cells were transduced with integrating lentiviral vector at MOI=5 or IDLVs at indicated MOIs in the presence of 5 μg/ml rapamycin. Percentage of GFP expression, mean fluorescent intensity, and provirus copy numbers were assessed by flow cytometry and qPCR for (A-C) integrating vector and (D-F) IDLV transductions.

FIGS. 5A-5C show that rapamycin does not increase lentiviral transduction efficiency in myeloid or T cells. (A) Primary human blood monocytes, monocyte-derived dendritic cells (MDDC) and macrophages (MDmac) were transduced at MOI=50 in the presence of indicated concentrations of rapamycin, and GFP expression was assessed by flow cytometry 13 days post transduction. (B) Primary human resting CD4⁺ T cells and (C) activated CD4⁺ T cells were transduced at indicated MOIs and rapamycin concentrations, and GFP expression was assessed by flow cytometry 9 days post transduction.

FIGS. 6A-6C show that rapamycin is required early for increased transduction efficiency. (A) Stimulated cord blood CD34⁺ cells were treated with 20 μg/ml rapamycin for various durations (indicated by red arrows), either before or after the start of transduction. (B) CD34+ cells were pre-treated with rapamycin, washed, and transduced with MOI=25. (C) CD34⁺ cells were transduced with MOI=50, and treated with rapamycin concurrent with or after the start of transduction. Percentages of GFP+ cells 11-14 days post transduction were assessed by flow cytometry. Line represents mean of duplicate or triplicate transductions.

FIGS. 7A-7G show that rapamycin increases vector entry and subsequent reverse transcription. (A) Entry of HIV-1 vectors carrying BLAM-Vpr fusion proteins into stimulated cord blood CD34⁺ cells was determined by the percentage of cells containing cleaved BLAM substrate. (B) HIV-1 strong-stop DNA, (C) full-length DNA, and (D) 2-LTR circles in stimulated human cord blood CD34⁺ cells, transduced with MOI=25, were quantified by qPCR at indicated time points after the start of transduction. Ratios of (E) HIV-1 full-length to strong-stop DNA and (F) 2-LTR circles to full-length DNA are shown as percentages. Data are representative of two separate experiments. (G) HIV-1 strong-stop (early RT) and full-length DNA (late RT) in stimulated human cord blood CD34⁺ cells, transduced with integrase-defective lentiviral vectors (IDLVs) at MOI=50, were quantified by qPCR at 12 h post transduction.

FIGS. 8A-8B show that autophagy induction, but not autophagosome accumulation, is required for efficient transduction. Stimulated cord blood CD34⁺ cells were transduced in the presence of (A) 3-methyladenine, an autophagy inhibitor, or (B) bafilomycin A1 (Baf) or chloroquine (CQ), molecules that cause accumulation of autophagosomes by inhibiting lysosomal fusion and acidifaction. Baf and CQ were added simultaneously with the vector or delayed by 2 or 6 hours, in order to allow endocytic entry of VSV-G pseudotyped vectors. Line represents mean of duplicate transductions.

FIG. 9 shows that rapamycin treatment does not alter the cell cycle distribution of HSCs. Human cord blood CD34⁺ HSCs were pre-stimulated and treated with or without rapamycin, and (a) DNA content and (b) RNA content were analyzed by Hoechst 33258 and pyronin Y staining, respectively. Red, no drug; blue, DMSO-treated; green, rapamycin-treated.

FIG. 10 shows that rapamycin treatment increases p21 mRNA levels. Stimulated cord blood CD34⁺ cells were treated with rapamycin (20 μg/ml) for 6 hours, and p21 mRNA levels were quantified by RT-PCR. Line represents mean of two independent experiments.

FIG. 11 shows that transduction efficiency of lenviral vector into stem cells is enhanced in the presence of mTOR inhibitor Torin 1.

FIG. 12 shows that transduction efficiency of LASV-pseudotyped vector into stem cells is also enhanced by rapamycin treatment.

### DETAILED DESCRIPTION OF THE INVENTION

### I. Overview

The present invention is predicated in part on the discoveries by the present inventors that inhibition of host cell mTOR complexes (via, e.g., allosteric mTOR inhibitor rapamycin or ATP-competitive mTOR inhibitor Torin 1) can enhance efficiency of retroviral transduction into stem cells. By relieving resistance to lentiviral vector entry and integration in human and mouse hematopoietic stem cells, this allows high frequency targeting of stem cells (more cells targeted) and effective delivery of payload (more product per cell). As detailed in the Examples herein, the inventors treated ex vivo adult or cord blood derived CD34⁺ cells, the cell population containing human hematopoietic stem cells, in the presence of an inhibitor of mTOR complexes (e.g., rapamycin) and lentiviral vectors containing the EGFP reporter gene. High frequency targeting and efficient delivery was then evident from EGFP gene marking. To ensure that hematopoietic stem cells were the marked cell population, the inventors utilized humanized immunodeficient mice (the current gold standard in animal models for human stem cell readout) to demonstrate that high frequencies of gene marked human cells.

To provide additional confirmation that stem cells were marked, human stem cells obtained from human stem cell engrafted mice were removed and transferred to new mouse recipients (secondary recipients) not containing human stem cells. These humanized mice gave rise to >90% EGFP-marked human cell populations over time. Since only human stem hematopoietic cells give rise to progeny in the secondary mouse recipients, the studies demonstrated that human hematopoietic stem cells were >90% EGFP-marked, which is 4-5 fold higher than that of other known methods of treatment to increase the frequency of gene marking. Importantly, rapamycin also shows the same effects on mouse stem and early progenitor cells which indicate that the effects are not restricted to human cells and can be universal for primate and nonprimate hematopoietic stem cells. The inventors also observed that other mTORs inhibitors (e.g., Torin 1) can also enhance lentiviral transduction, similar to what was achieved with allosteric inhibitor rapamycin. Further, it was found that enhanced retroviral transduction mediated by mTOR inhibition is not limited to a specific viral entry mechanism but is instead applicable to multiple endocytic entry mechanisms with distinct receptor usage.

In accordance with these studies, the present invention provides methods for using inhibitors of mTOR complexes (e.g., mTOR kinase inhibitor such as rapamycin and functional derivatives, variants or analog compounds of rapamycin, as well as other mTOR inhibitors described herein) to promote high frequency targeting and efficient payload delivery to a target host cell (e.g., human and mouse hematopoietic stem cells). Methods of the invention allow very efficient, e.g., a 4-5 fold increase over the current state of the art methods for viral vector delivery to hematopoietic stem cells. With the present invention, efficient viral vector-mediated delivery to stem cells can be achieved with reduced amounts of viral vectors for treatment, thus decreasing the probability of insertional mutagenesis. In addition, increased viral vector entry per hematopoietic stem cell or progenitor cell allows treatment with non-integrating vectors which can be used for enhanced gene repair without the ensuing gene insertional problems. Moreover, the short length of culture time for the enhanced entry/transduction effect ensures that the hematopoietic stem cells don't differentiate, thus remaining stem cells with the capacity to home to their appropriate environment. Finally, employing an inhibitor of mTOR complexes (e.g., rapamycin or related compound) in viral transduction will both reduce the cost of hematopoietic stem cell transduction and increase the yield.

The methods of the invention are applicable to enhancing transduction efficiency of retroviral vectors (including lentiviral vectors) into various host cells. In some preferred embodiments, the methods are employed for retroviral transduction into stem cells. Suitable stem cells are not limited to any specific hematopoietic stem cell gestational age or specific species. As exemplified herein, the methods of the invention are suitable for different stem cells including, e.g., cord blood or adult human stem and progenitor cells as well as comparable cells from mice.

### II. Definition

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). In addition, the following definitions are provided to assist the reader in the practice of the invention.

The term “analog” is used herein to refer to a molecule that structurally resembles a reference molecule but which has been modified in a targeted and controlled manner, by replacing a specific substituent of the reference molecule with an alternate substituent. Compared to the reference molecule (e.g., rapamycin), an analog can exhibit the same, similar, or improved utility. Methods for synthesizing and screening candidate analog compounds of a reference molecule to identify analogs having altered or improved traits (e.g., a rapamycin analog compound with enhanced inhibitory activity than rapamycin on lymphocyte response to IL-2) are well known in the art.

The term “contacting” has its normal meaning and refers to combining two or more agents (e.g., two compounds or a compound and a cell) or combining agents and cells. Contacting can occur in vitro, e.g., mixing a compound and a cultured cell in a test tube or other container. It can also occur in vivo (contacting a compound with a cell within a subject) or ex vivo (contacting the cell with compound outside the body of a subject and followed by introducing the treated cell back into the subject).

Host cell restriction refers to resistance or defense of cells against viral infections. Mammalian cells can resist viral infections by a variety of mechanisms. Viruses must overcome host cell restrictions to successfully reproduce their genetic material.

Retroviruses are enveloped viruses that belong to the viral family Retroviridae. The virus itself stores its nucleic acid, in the form of a +mRNA (including the 5′-cap and 3′-PolyA inside the virion) genome and serves as a means of delivery of that genome into host cells it targets as an obligate parasite, and constitutes the infection. Once in a host's cell, the virus replicates by using a viral reverse transcriptase enzyme to transcribe its RNA into DNA. The DNA is then integrated into the host's genome by an integrase enzyme. The retroviral DNA replicates as part of the host genome, and is referred to as a provirus. Retroviruses include the genus of Alpharetrovirus (e.g., avian leukosis virus), the genus of Betaretrovirus; (e.g., mouse mammary tumor virus), the genus of Gammaretrovirus (e.g., murine leukemia virus or MLV), the genus of Deltaretrovirus (e.g., bovine leukemia virus and human T-lymphotropic virus), the genus of Epsilonretrovirus (e.g., Walleye dermal sarcoma virus), and the genus of Lentivirus.

Lentivirus is a genus of viruses of the Retroviridae family, characterized by a long incubation period. Lentiviruses can deliver a significant amount of genetic information into the DNA of the host cell, so they are one of the most efficient methods of a gene delivery vector. Examples of lentiviruses include human immunodeficiency viruses (HIV-1 and HIV-2), simian immunodeficiency virus (SIV), and feline immunodeficiency virus (FIV). Additional examples include BLV, EIAV and CEV.

mTOR, or the “mammalian target of rapamycin,” is a protein that in humans is encoded by the FRAP1 gene. mTOR is a serine/threonine protein kinase that regulates cell growth, cell proliferation, cell motility, cell survival, protein synthesis, and transcription. mTOR, which belongs to the phosphatidylinositol 3-kinase-related kinase protein family, is the catalytic subunit of two molecular complexes: mTORC1 and mTORC2.

mTOR Complex 1 (mTORC1) is composed of mTOR, regulatory-associated protein of mTOR (Raptor), mammalian lethal with SEC 13 protein 8 (MLST8) and partners PRAS40 and DEPTOR. This complex is characterized by the classic features of mTOR by functioning as a nutrient/energy/redox sensor and controlling protein synthesis. The activity of this complex is stimulated by insulin, growth factors, serum, phosphatidic acid, amino acids (particularly leucine), and oxidative stress. mTOR Complex 2 (mTORC2) is composed of mTOR, rapamycin-insensitive companion of mTOR (RICTOR), GOL, and mammalian stress-activated protein kinase interacting protein 1 (mSINI). mTORC2 has been shown to function as an important regulator of the cytoskeleton through its stimulation of F-actin stress fibers, paxillin, RhoA, Rac1, Cdc42, and protein kinase C α (PKCα). mTORC2 also appears to possess the activity of a previously elusive protein known as “PDK2”. mTORC2 phosphorylates the serine/threonine protein kinase Akt/PKB at a serine residue S473.

The term “mutagenesis” or “mutagenizing” refers to a process of introducing changes (mutations) to the base pair sequence of a coding polynucleotide sequence and consequential changes to its encoded polypeptide. Unless otherwise noted, the term as used herein refers to mutations artificially introduced to the molecules as opposed to naturally occurring mutations caused by, e.g., copying errors during cell division or that occurring during processes such as meiosis or hypermutation. Mutagenesis can be achieved by a number of means, e.g., by exposure to ultraviolet or ionizing radiation, chemical mutagens, or viruses. It can also be realized by recombinant techniques such as site-specific mutagenesis, restriction digestion and religation, error-prone PCR, polynucleotide shuffling and etc. For a given polynucleotide encoding a target polypeptide, mutagenesis can result in mutants or variants that contain various types of mutations, e.g., point mutations (e.g., silent mutations, missense mutations and nonsense mutations), insertions, or deletions.

The term “operably linked” when referring to a nucleic acid, means a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame.

The term “polynucleotide” or “nucleic acid” as used herein refers to a polymeric form of nucleotides of any length, either ribonucleotides or deoxyribonucleotides, that comprise purine and pyrimidine bases, or other natural, chemically or biochemically modified, non-natural, or derivatized nucleotide bases. Polynucleotides of the embodiments of the invention include sequences of deoxyribopolynucleotide (DNA), ribopolynucleotide (RNA), or DNA copies of ribopolynucleotide (cDNA) which may be isolated from natural sources, recombinantly produced, or artificially synthesized. A further example of a polynucleotide is polyamide polynucleotide (PNA). The polynucleotides and nucleic acids may exist as single-stranded or double-stranded. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. The polymers made of nucleotides such as nucleic acids, polynucleotides and polynucleotides may also be referred to herein as nucleotide polymers.

Polypeptides are polymer chains comprised of amino acid residue monomers which are joined together through amide bonds (peptide bonds). The amino acids may be the L-optical isomer or the D-optical isomer. In general, polypeptides refer to long polymers of amino acid residues, e.g., those consisting of at least more than 10, 20, 50, 100, 200, 500, or more amino acid residue monomers. However, unless otherwise noted, the term polypeptide as used herein also encompass short peptides which typically contain two or more amino acid monomers, but usually not more than 10, 15, or 20 amino acid monomers.

Proteins are long polymers of amino acids linked via peptide bonds and which may be composed of two or more polypeptide chains. More specifically, the term “protein” refers to a molecule composed of one or more chains of amino acids in a specific order; for example, the order as determined by the base sequence of nucleotides in the gene coding for the protein. Proteins are essential for the structure, function, and regulation of the body's cells, tissues, and organs, and each protein has unique functions. Examples are hormones, enzymes, and antibodies. In some embodiments, the terms polypeptide and protein may be used interchangeably.

Stem cells are biological cells found in all multicellular organisms, and can divide (through mitosis) and differentiate into diverse specialized cell types and can self-renew to produce more stem cells. In mammals, there are two broad types of stem cells: embryonic stem cells, which are isolated from the inner cell mass of blastocysts, and adult stem cells, which are found in various tissues. In adult organisms, stem cells and progenitor cells act as a repair system for the body, replenishing adult tissues. In a developing embryo, stem cells can differentiate into all the specialized cells (these are called pluripotent cells), but also maintain the normal turnover of regenerative organs, such as blood, skin, or intestinal tissues. There are three accessible sources of autologous adult stem cells in humans: bone marrow, adipose tissue (lipid cells) and blood. Stem cells can also be taken from umbilical cord blood just after birth.

Hematopoietic stem cells (HSCs) are a heterogeneous population of multipotent stem cells that can give rise to all the blood cell types from the myeloid (monocytes and macrophages, neutrophils, basophils, eosinophils, erythrocytes, megakaryocytes/platelets, dendritic cells), and lymphoid lineages (T-cells, B-cells, NK-cells). These cells are found in the bone marrow of adults; within femurs, pelvis, ribs, sternum, and other bones. The cells can usually be obtained directly from the iliac crest part of the pelvic bone, using a special needle and a syringe. They are also collected from the peripheral blood following pre-treatment with cytokines, such as G-CSF (granulocyte colony-stimulating factors) or other reagents that induce cells to be released from the bone marrow compartment. Other sources for clinical and scientific use include umbilical cord blood, as well as peripheral blood.

A cell has been “transformed” or “transfected” by exogenous or heterologous polynucleotide when such polynucleotide has been introduced inside the cell. The transforming polynucleotide may or may not be integrated (covalently linked) into the genome of the cell. In prokaryotes, yeast, and mammalian cells for example, the transforming polynucleotide may be maintained on an episomal element such as a plasmid. With respect to eukaryotic cells, a stably transformed cell is one in which the transforming polynucleotide has become integrated into a chromosome so that it is inherited by daughter cells through chromosome replication. This stability is demonstrated by the ability of the eukaryotic cell to establish cell lines or clones comprised of a population of daughter cells containing the transforming polynucleotide. A “clone” is a population of cells derived from a single cell or common ancestor by mitosis. A “cell line” is a clone of a primary cell that is capable of stable growth in vitro for many generations.

A “variant” of a reference molecule (e.g., rapamycin) refers to a molecule which has a structure that is derived from or similar to that of the reference molecule. Typically, the variant is obtained by modification of the reference molecule in a controlled or random manner. As detailed herein, methods for modifying a reference molecule to obtain functional derivative compounds that have similar or improved properties relative to that of the reference molecule are well known in the art.

A “vector” is a replicon, such as plasmid, phage or cosmid, to which another polynucleotide segment may be attached so as to bring about the replication of the attached segment. Vectors capable of directing the expression of genes encoding for one or more polypeptides are referred to as “expression vectors”.

A retrovirus (e.g., a lentivirus) based vector or retroviral vector means that genome of the vector comprises components from the virus as a backbone. The viral particle generated from the vector as a whole contains essential vector components compatible with the RNA genome, including reverse transcription and integration systems. Usually these will include the gag and pol proteins derived from the virus. If the vector is derived from a lentivirus, the viral particles are capable of infecting and transducing non-dividing cells. Recombinant retroviral particles are able to deliver a selected exogenous gene or polynucleotide sequence such as therapeutically active genes, to the genome of a target cell.

III. Inhibitors of mTOR Complexes Suitable for the Invention

The present invention relates to novel methods and compositions for high frequency targeting and efficient payload delivery of viral vectors to host cells. The invention is based on the discovery by the present inventors that inhibition of signaling of host cell mTOR complexes allows for more efficient viral transduction into the host cell. “Inhibitors of mTOR complexes” (or “mTOR complex inhibitors”) suitable for the invention are any compounds that inhibit or antagonize one or both of the mTOR complexes, mTORC1 and/or mTORC2. These include compounds that inhibit the mTOR kinase, as well as compounds that otherwise suppress or antagonize signaling activities of the mTOR complexes or negatively affect their biological properties (e.g., destabilizing or disrupting the protein complexes). For example, they can be compounds that do not directly impact the mTOR kinase, but through other components of the mTOR protein complexes (e.g., Raptor or RICTOR) can disrupt, or inhibit the formation of, the mTORC1 complex and/or the mTORC2 complex or inhibit interaction of the complexes with downstream signaling molecules.

In some embodiments of the invention, the employed inhibitor is a compound that antagonizes the mTOR kinase (mTOR inhibitors). Various mTOR inhibitors known in the art can be employed in the practice of the present invention. As used herein, the term “mTOR inhibitor” or “mTOR inhibitor compound” broadly encompasses any compounds that directly or indirectly inhibit or antagonize mTOR biological activities (e.g., kinase activity) or mTOR mediated signaling activities. Thus, the mTOR inhibitor can be a compound that suppresses mTOR expression or affects its cellular stability, a compound that inhibits or prevents formation of mTOR complexes, a compound that inhibits mTOR binding to its intracellular receptor FKBP12, a compound that inhibits or antagonizes enzymatic activities of mTOR, or a compound that otherwise inhibits mTOR interaction with downstream molecules.

Some embodiments of the invention employ rapamycin. Rapamycin (Vezina et al., J. Antibiot. 1975; 28: 721\u20136), also known as Sirolimus, is an immunosuppressant drug used to prevent rejection in organ transplantation. It prevents activation of T cells and B-cells by inhibiting their response to interleukin-2 (IL-2). It was approved by the FDA in September 1999 and is marketed under the trade name Rapamune by Pfizer. Rapamycin is an allosteric mTOR inhibitor. Other than rapamycin, any compounds that specifically mimic or enhance the biological activity of rapamycin (e.g., binding to the FKBP12-rapamycin-binding domain of mTOR and/or inhibiting mTOR kinase activity) can be used in the invention. For example, mTOR is the principal cellular target of rapamycin. Thus, rapamycin analogs or functional derivatives with similar or improved inhibitory activity on mTOR may be suitable for the present invention. These include rapamycin analog compounds known in the art. Examples include compounds described in, e.g., Ritacco et al., Appl Environ Microbiol. 2005; 71: 1971-1976; Bayle et al., Chemistry & Biology 2006; 13: 99-107; Wagner et al., Bioorg Med Chem Lett. 2005; 15:5340-3; Graziani et al., Org Lett. 2003; 5:2385-8; Ruan et al., Proc. Natl. Acad. Sci. USA 2008; 105:33-8; U.S. Pat. No. 5,138,051; and WO/2009/131631. Several semi-synthetic rapamycin analogs (also known as rapalogues) have been evaluated by pharmaceutical companies for clinical development, e.g., temsirolimus (CCI-779, Torisel, Wyeth Pharmaceuticals), everolimus (RAD001, Afinitor, Novartis Pharmaceuticals), and ridaforolimus (AP23573; formerly deforolimus, ARIAD Pharmaceuticals).

Some other embodiments of the invention can employ ATP-competitive mTOR inhibitors. These mTOR inhibitors are ATP analogues that inhibit mTOR kinase activity by competing with ATP for binding to the kinase domain in mTOR. Unlike rapamycin, which primarily inhibits only mTORC1, the ATP analogues inhibit both mTORC1 and mTORC2. Because of the similarity between the kinase domains of mTOR and the PI3Ks, mTOR inhibition by some of these compounds overlaps with PI3K inhibition. Some of the ATP-competitive inhibitors are dual mTOR/PI3K inhibitors (which inhibit both kinases at similar effective concentrations). Examples of such inhibitors include PI103, PI540, PI620, NVP-BEZ235, GSK2126458, and XL765. These compounds are all well known in the art. See, e.g., Fan et al., Cancer Cell 9:341-349, 2006; Raynaud et al., Mol. Cancer Ther. 8:1725-1738, 2009; Maira et al., Mol. Cancer Ther. 7: 1851-63, 2008; Knight et al., ACS Med. Chem. Lett., 1: 39-43, 2010; and Prasad et al., Neuro. Oncol. 13: 384-92, 2011. Some other ATP-competitive mTOR inhibitors are more selective for mTOR (pan-mTOR inhibitors) which have an IC50 for mTOR inhibition that is significantly lower than that for PI3K. These include, e.g., PP242, INKI28, AZD8055, AZD2014, OSI027, TORKi CC223; and Palomid 529. These compounds have also been structurally and functionally characterized in the art. See, e.g., Apsel et al., Nature Chem. Biol. 4: 691-9, 2008; Jessen et al., Mol. Cancer Ther. 8 (Suppl. 12), Abstr. B 148, 2009; Pike et al., Bioorg. Med. Chem. Lett. 23:1212-6, 2013; Bhagwat et al., Mol. Cancer Ther. 10:1394-406, 2011; and Xue et al., Cancer Res. 68: 9551-7, 2008.

Additional ATP-competitive mTOR inhibitors that can be employed in the present invention include, e.g., WAY600, WYE354, WYE687, and WYE125132. See, e.g., Yu et al., Cancer Res. 69: 6232-40, 2009; and Yu et al., Cancer Res. 70: 621-31, 2010. These compounds all have greater selectivity for mTORC1 and mTORC2 over PI3K. They are derived from WAY001, which is a lead compound identified from a high-throughput screen directed against recombinant mTOR and which is more potent against PI3K than against mTOR. Various other mTOR inhibitors known in the art can also be used in the practice of the present invention. These include, e.g., Torin 1 (Thoreen et al., J. Biol. Chem. 284: 8023-32, 2009), Torin2 (Liu et al., J. Med. Chem. 54:1473-80, 2011), Ku0063794 (Garcia-Martinez et al., Biochem. J. 421: 29-42, 2009), WJD008 (Li et al., J. Pharmacol. Exp. Ther. 334: 830-8, 2010), PKI402 (Mallon et al., Mol. Cancer Ther. 9: 976-84, 2010), NVP-BBD130 (Marone et al., Mol. Cancer Res. 7: 601-13, 2009), NVP-BAG956 (Marone et al., Mol. Cancer Res. 7: 601-13, 2009), and OXA-01 (Falcon et al., Cancer Res. 71: 1573-83, 2011).

Other than mTOR inhibitors that bind to and directly inhibit mTORC1 and/or mTORC2 complexes, compounds which antagonize mTOR activities in other manners may also be employed in the practice of the present invention. These include, e.g., Metformin which indirectly inhibits mTORC1 through activation of AMPK; compounds which are capable of targeted disruption of the multiprotein TOR complexes formed from mTORC1 and mTORC2, e.g., nutlin 3 and ABT-263 (Secchiero et al., Curr. Pharm. Des. 17, 569-77, 2011; and Tse et al., Cancer Res. 68: 3421-8, 2008); compounds which antagonize or inhibit phosphatidic acid mediated activation of mTORs, e.g., HTS-1 (Veverka et al., Oncogene 27: 585-95, 2008); and compounds which block the activity of mTORC1 activator RHEB, e.g., farnesylthiosalicylic acid (McMahon et al., Mol. Endocrinol. 19:175-83, 2005).

Suitable compounds for the invention also include novel inhibitors of mTOR complexes or mTOR inhibitors (e.g., other rapamycin analogs) that can be identified in accordance with screening assays routinely practiced in the art. For example, a library of candidate compounds can be screened in vitro for mTOR inhibitors or rapamycin derivatives that inhibit mTOR. This can be performed using methods as described in, e.g., Yu et al., Cancer Res. 69: 6232-40, 2009; Livingstone et al., Chem Biol. 2009, 16:1240-9; Chen et al., ACS Chem Biol. 2012, 7:715-22; and Bhagwat et al., Assay Drug Dev Technol. 2009, 7:471-8. The candidate compounds can be randomly synthesized chemical compounds, peptide compounds or compounds of other chemical nature. The candidate compounds can also comprise molecules that are derived structurally from known mTOR inhibitors described herein (e.g., rapamycin or analogs).

The various inhibitors of mTOR complexes (e.g., mTOR inhibitors) described herein can be readily obtained from commercial sources. For example, rapamycin, some rapalogues described herein, and various ATP-competitive mTOR inhibitors (e.g., Torin 1) can be purchased from a number of commercial suppliers. These include, e.g., EMD Chemicals, R&D Systems, Sigma-Aldrich, MP Biomedicals, Enzo Life Sciences, Santa Cruz Biotech, and Invitrogen. Alternatively, the inhibitors of mTOR complexes can be generated by de novo synthesis based on teachings in the art via routinely practiced protocols of organic chemistry and biochemistry. For example, methods for synthesizing rapamycin are described in the art, e.g., Ley et al., Chemistry. 2009;15:2874-914; Nicolaou et al., J. Am. Chem. Soc. 1993, 115: 4419; Hayward et al., J. Am. Chem. Soc. 1993, 115: 9345; Romo et al., J. Am. Chem. Soc. 1993, 115: 7906; Smith et al., J. Am. Chem. Soc. 1995, 117: 5407-5408; and Maddess et al., Angew. Chem. Int. Ed. 2007, 46, 591. Structures and chemical synthesis of various other mTOR inhibitors suitable for the invention are also well characterized in the art.

IV. Enhancing Viral Transduction by Inhibiting Host Cell mTOR Complexes

The invention provides methods and compositions for enhanced viral transduction into the host cell. The methods of the present invention can be used to enhance transduction efficiency of recombinant retroviruses or retroviral vectors expressing various exogenous genes. For example, recombinant retroviruses expressing an exogenous gene or heterologous polynucleotide sequence can be transduced into host cells with enhanced transduction efficiency in various gene therapy and agricultural bioengineering applications. In some preferred embodiments, the methods are intended for enhanced viral transduction in gene therapy. For example, a current problem with clinical stem cell based therapy is that viral vector entry and payload delivery does not occur without some form of stem cell proliferation. This potentially can result in differentiation of stem cells and loss of stem cell function when placed back into the host. Employing inhibitors of mTOR complexes (e.g., mTOR inhibitors such as rapamycin), the invention provides methods for enhancing transduction of recombinant vectors, esp. retroviral vectors. Methods of the invention allow high frequency targeting to stem cells, and high efficiency delivery, without overt stem cell engraftment and growth problems.

Typically, methods of the invention involve transfecting a retroviral vector into a host cell (e.g., a stem cell such as human HSCs) in the presence of a suitable amount of an inhibitor of mTOR complexes (e.g., mTOR inhibitors such as rapamycin). The inhibitor of mTOR complexes can be contacted with the cell prior to, simultaneously with, or subsequent to addition of the retroviral vector or recombinant retrovirus. This is followed by culturing the host cells under suitable conditions so that the viral vector or virus can be transduced into the cells.

Methods of the invention can be employed for enhancing transduction efficiency of various recombinant viruses or viral vectors used for gene transfer in many settings. In some embodiments, methods of the invention are used for promoting transduction of retroviruses or retroviral vectors, e.g., lentiviral vectors. Retroviruses are a group of single-stranded RNA viruses characterized by an ability to convert their RNA to double-stranded DNA in infected cells by a process of reverse-transcription. The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These elements contain strong promoter and enhancer sequences and are also required for integration in the host cell genome.

Retroviral vectors or recombinant retroviruses are widely employed in gene transfer in various therapeutic or industrial applications. For example, gene therapy procedures have been used to correct acquired and inherited genetic defects, and to treat cancer or viral infection in a number of contexts. The ability to express artificial genes in humans facilitates the prevention and/or cure of many important human diseases, including many diseases which are not amenable to treatment by other therapies. For a review of gene therapy procedures, see Anderson, Science 256:808-813, 1992; Nabel & Feigner, TIBTECH 11:211-217, 1993; Mitani & Caskey, TIBTECH 11:162-166, 1993; Mulligan, Science 926-932, 1993; Dillon, TIBTECH 11:167-175, 1993; Miller, Nature 357:455-460, 1992; Van Brunt, Biotechnology 6:1149-1154, 1998; Vigne, Restorative Neurology and Neuroscience 8:35-36, 1995; Kremer & Perricaudet, British Medical Bulletin 51:31-44, 1995; Haddada et al., in Current Topics in Microbiology and Immunology (Doerfler & Böhm eds., 1995); and Yu et al., Gene Therapy 1:13-26, 1994.

In order to construct a retroviral vector for gene transfer, a nucleic acid encoding a gene of interest is inserted into the viral genome in the place of certain viral sequences to produce a viral construct that is replication-defective. In order to produce virions, a producer host cell or packaging cell line is employed. The host cell usually expresses the gag, pol, and env genes but without the LTR and packaging components. When the recombinant viral vector containing the gene of interest together with the retroviral LTR and packaging sequences is introduced into this cell line (e.g., by calcium phosphate precipitation), the packaging sequences allow the RNA transcript of the recombinant vector to be packaged into viral particles, which are then secreted into the culture media. The media containing the recombinant retroviruses is then collected, optionally concentrated, and used for transducing host cells (e.g., stem cells) in gene transfer applications.

Suitable host or producer cells for producing recombinant retroviruses or retroviral vectors according to the invention are well known in the art (e.g., 293T cells exemplified herein). Many retroviruses have already been split into replication defective genomes and packaging components. For other retroviruses, vectors and corresponding packaging cell lines can be generated with methods routinely practiced in the art. The producer cell typically encodes the viral components not encoded by the vector genome such as the gag, pol and env proteins. The gag, pol and env genes may be introduced into the producer cell and stably integrated into the cell genome to create a packaging cell line. The retroviral vector genome is then introduced-into the packaging cell line by transfection or transduction to create a stable cell line that has all of the DNA sequences required to produce a retroviral vector particle. Another approach is to introduce the different DNA sequences that are required to produce a retroviral vector particle, e.g. the env coding sequence, the gag-pol coding sequence and the defective retroviral genome into the cell simultaneously by transient triple transfection. Alternatively, both the structural components and the vector genome can all be encoded by DNA stably integrated into a host cell genome.

The methods of the invention can be practiced with various retroviral vectors and packaging cell lines well known in the art. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), simian immunodeficiency virus (SW), human immunodeficiency virus (HIV), and combinations thereof (see, e.g., Buchscher et al., J. Virol. 66:2731-2739, 1992; Johann et al., J. Virol. 66:1635-1640, 1992; Sommerfelt et al., Virol. 176:58-59, 1990; Wilson et al., J. Virol. 63:2374-2378, 1989; Miller et al., J. Virol. 65:2220-2224, 1991; and PCT/US94/05700). Particularly suitable for the present invention are lentiviral vectors. Lentiviral vectors are retroviral vector that are able to transducer or infect non-dividing cells and typically produce high viral titers. Lentiviral vectors have been employed in gene therapy for a number of diseases. For example, hematopoietic gene therapies using lentiviral vectors or gamma retroviral vectors have been used for x-linked adrenoleukodystrophy and beta thalassaemia. See, e.g., Kohn et al., Clin. Immunol. 135:247-54, 2010; Cartier et al., Methods Enzymol. 507:187-198, 2012; and Cavazzana-Calvo et al., M, Payen E, Negre O, et al. Transfusion independence and HMGA2 activation after gene therapy of human beta-thalassaemia. Nature 467:318-322, 2010. Methods of the invention can be readily applied in gene therapy or gene transfer with such vectors. In some other embodiments, other retroviral vectors can be used in the practice of the methods of the invention. These include, e.g., vectors based on human foamy virus (HFV) or other viruses in the Spumavirus genera.

In particular, a number of viral vector approaches are currently available for gene transfer in clinical trials, with retroviral vectors by far the most frequently used system. All of these viral vectors utilize approaches that involve complementation of defective vectors by genes inserted into helper cell lines to generate the transducing agent. pLASN and MFG-S are examples are retroviral vectors that have been used in clinical trials (Dunbar et al., Blood 85:3048-305 (1995); Kohn et al., Nat. Med. 1:1017-102 (1995); Malech et al., Proc. Natl. Acad. Sci. U.S.A. 94:22 12133-12138 (1997)). PA317/pLASN was the first therapeutic vector used in a gene therapy trial. (Blaese et al., Science 270:475-480, 1995). Transduction efficiencies of 50% or greater have been observed for MFG-S packaged vectors (Ellem et al., Immunol Immunother. 44:10-20, 1997; Dranoff et al., Hum. Gene Ther. 1:111-2, 1997). Many producer cell line or packaging cell line for transfecting retroviral vectors and producing viral particles are also known in the art. The producer cell to be used in the invention needs not to be derived from the same species as that of the target cell (e.g., human target cell). Instead, producer or packaging cell lines suitable for the present invention include cell lines derived from human (e.g., HEK 292 cell), monkey (e.g., COS-1cell), mouse (e.g., NIH 3T3 cell) or other species (e.g., canine). Some of the cell lines are disclosed in the Examples below. Additional examples of retroviral vectors and compatible packaging cell lines for producing recombinant retroviruses in gene transfers are reported in, e.g., Markowitz et al., Virol. 167:400-6, 1988; Meyers et al., Arch. Virol. 119:257-64, 1991 (for spleen necrosis virus (SNV)-based vectors such as vSNO21); Davis et al., Hum. Gene. Ther. 8:1459-67, 1997 (the “293-SPA” cell line); Povey et al., Blood 92:4080-9, 1998 (the “1MI-SCF” cell line); Bauer et al., Biol. Blood Marrow Transplant. 4:119-27, 1998 (canine packaging cell line “DA”); Gerin et al., Hum. Gene Ther. 10:1965-74, 1999; Sehgal et al., Gene Ther. 6:1084-91, 1999; Gerin et al., Biotechnol. Prog. 15:941-8, 1999; McTaggart et al., Biotechnol. Prog. 16:859-65, 2000; Reeves et al., Hum. Gene. Ther. 11:2093-103, 2000; Chan et al., Gene Ther. 8:697-703, 2001; Thaler et al., Mol. Ther. 4:273-9, 2001; Martinet et al., Eur. J. Surg. Oncol. 29:351-7, 2003; and Lemoine et al., I. Gene Med. 6:374-86, 2004. Any of these and other retroviral vectors and packaing producer cell lines can be used in the practice of the present invention.

Many of the retroviral vectors and packing cell lines used for gene transfer in the art can be obtained commercially. For example, a number of retroviral vectors and compatible packing cell lines are available from Clontech (Mountain View, Calif.). Examples of lentiviral based vectors include, e.g., pLVX-Puro, pLVX-IRES-Neo, pLVX-IRES-Hyg, and pLVX-IRES-Puro. Corresponding packaging cell lines are also available, e.g., Lenti-X 293T cell line. In addition to lentiviral based vectors and packaging system, other retroviral based vectors and packaging systems are also commercially available. These include MMLV based vectors pQCXIN, pQCXIQ and pQCXIH, and compatible producer cell lines such as HEK 293 based packaging cell lines GP2-293, EcoPack 2-293 and AmphoPack 293, as well as NIH/3T3-based packaging cell line RetroPack PT67. Any of these and other retroviral vectors and producer cell lines may be employed in the practice of the present invention.

The methods of the invention can be employed in the transfer and recombinant expression of various exogenous genes or heterologous polynucleotide sequences. Typically, the gene or heterologous polynucleotide sequence is derived from a source other than the retroviral genome which provides the backbone of the vector used in the gene transfer. The gene may be derived from a prokaryotic or eukaryotic source such as a bacterium, a virus, a yeast, a parasite, a plant, or an animal. The exogenous gene or heterologous polynucleotide sequence expressed by the recombinant retroviruses can also be derived from more than one source, i.e., a multigene construct or a fusion protein. In addition, the exogenous gene or heterologous polynucleotide sequence may also include a regulatory sequence which may be derived from one source and the gene from a different source. For any given gene to be transferred via the viral vectors, a recombinant retroviral vector can be readily constructed by inserting the gene operably into the vector, replicating the vector in an appropriate packaging cell as described above, obtaining viral particles produced therefrom, and then infecting target cells (e.g., stem cells) with the recombinant viruses.

In some preferred embodiments, the exogenous gene or heterologous polynucleotide sequence harbored by the recombinant retrovirus is a therapeutic gene. The therapeutic gene can be transferred, for example to treat cancer cells, to express immunomodulatory genes to fight viral infections, or to replace a gene's function as a result of a genetic defect. The exogenous gene expressed by the recombinant retrovirus can also encode an antigen of interest for the production of antibodies. In some exemplary embodiments, the exogenous gene to be transferred with the methods of the present invention is a gene that encodes a therapeutic polypeptide. For example, transfection of tumor suppressor gene p53 into human breast cancer cell lines has led to restored growth suppression in the cells (Casey et al., Oncogene 6:1791-7, 1991). In some other embodiments, the exogenous gene to be transferred with methods of the present invention encodes an enzyme. For example, the gene can encode a cyclin-dependent kinase (CDK). It was shown that restoration of the function of a wild-type cyclin-dependent kinase, pl6INK4, by transfection with a p16INK4-expressing vector reduced colony formation by some human cancer cell lines (Okamoto, Proc. Natl. Acad. Sci. U.S.A. 91:11045-9, 1994). Additional embodiments of the invention encompass transferring into target cells exogenous genes that encode cell adhesion molecules, other tumor suppressors such as p21 and BRCA2, inducers of apoptosis such as Bax and Bak, other enzymes such as cytosine deaminases and thymidine kinases, hormones such as growth hormone and insulin, and interleukins and cytokines.

The recombinant retroviruses or retroviral vectors expressing an exogenous gene can be transduced into any target cells in the presence of an inhibitor of mTOR complexes (e.g., an mTOR inhibitor such as an ATP-competitive inhibitor or allosteric inhibitor rapamycin) for recombinant expression of the exogenous gene. As exemplified herein, preferred target cells for the present invention are stem cells. Stem cells suitable for practicing the invention include and are not limited to hematopoietic stem cells (HSC), embryonic stem cells or mesenchymal stem cells. They include stem cells obtained from both human and non-human animals including vertebrates and mammals. Other specific examples of target cells include cells that originate from bovine, ovine, porcine, canine, feline, avian, bony and cartilaginous fish, rodents including mice and rats, primates including human and monkeys, as well as other animals such as ferrets, sheep, rabbits and guinea pigs.

Transducing a recombinant retroviral vector into the target cell in the presence of an inhibitor of mTOR complexes (e.g., rapamycin) can be carried out in accordance with protocols well known in the art or that exemplified in the Examples below. For example, the host cell (e.g., HSCs) may be pre-treated with the inhibitor compound prior to transfection with the retroviral vector. Alternatively, the target host cell can be transfected with the viral vector in the presence of an inhibitor of mTOR complexes described herein (e.g., rapamycin or an analog compound). The concentration of the inhibitor to be used can be easily determined and optimized by the skilled artisans, depending on the nature of the compound, the recombinant vector or virus used, as well as when the cell is contacted with the compound (prior to or simultaneously with transfection with the vector). Typically, the inhibitor (rapamycin or an analog) should present in a range from about 10 nM to about 2 mM. Preferably, the compound used in the methods is at a concentration of from about 50 nM to about 500 μM, from about 100 nM to 100 μM, or from about 0.5 μM to about 50 μM. More preferably, the compound is contacted with the producer cell at a concentration of from about 1 μM to about 20 μM, e.g., 1 μM, 2 μM, 5 μM or 10 μM.

The invention also provides pharmaceutical combinations, e.g. kits, that can be employed to carry out the various methods disclosed herein. Such pharmaceutical combinations typically contain an mTOR inhibitor compound (e.g., rapamycin or a rapamycin analog described herein), in free form or in a composition with one or more inactive agents, and other components. The pharmaceutical combinations can also contain one or more appropriate retroviral vectors (e.g., a lentiviral vector described herein) for cloning a target gene of interest. The pharmaceutical combinations can additionally contain a packaging or producer cell line (e.g., 293T cell line) for producing a recombinant retroviral vector that expresses an inserted target gene or polynucleotide of interest. In some embodiments, the pharmaceutical combinations contain a host cell or target cell into which an exogenous gene harbored by the recombinant retroviral vector or virus is to be delivered.

In various embodiments, the pharmaceutical combinations or kits of the invention can optionally further contain instructions or an instruction sheet detailing how to use the inhibitor of mTOR complexes (e.g., mTOR inhibitor such as rapamycin) to transduce recombinant retroviruses or retroviral vectors with enhanced efficiency.

### EXAMPLES

The following examples are provided to further illustrate the invention but not to limit its scope.

### Example 1 Materials and Methods

This Example describes some of the materials and methods employed in the studies described below.

Chemicals and Reagents. rapamycin, bafilomycin A1, and 3-methyladenine were obtained from Sigma-Aldrich. Rapamycin stock solution (2.5 ml/ml) was diluted to the final concentration (5-20 μg/ml) in the appropriate transduction mixture and was present throughout the 12 h transduction or at specified intervals. Bafilonycin A1 and 3-methyladenine were dissolved in DMSO and DMF, respectively. Chloroquine was obtained from Invitrogen as part of an LC3B antibody kit. All fluorescent antibodies for immunophenotyping were from BD and were used at a 1:50 dilution.

Vector production. HIV-1 vectors were produced by co-transfection of FG12 (10 μg), pMDLg/p (6.5 μg), VSV-G II (3.5 μg), and pRSV-Rev (2.5 μg) into 293T cells by calcium phosphate precipitation. Supernatant was concentrated by ultra-centrifugation at 194,000 rpm for 2.5 hours through a 20% sucrose cushion. Vector titer (TU/ml) was determined by transduction of 293T cells. For the production of BLAM-Vpr containing vector, pMM310 encoding a BLAM-Vpr fusion protein was co-transfected with the other plasmids at a 1:3 ratio to the transfer plasmid FG12.

HSC isolation and transduction. CD34⁺ HSCs were isolated from umbilical cord blood or adult bone marrow using the RosetteSep system according to manufacturer protocol (StemCell Technologies). The purity of CD34⁺ cell preparations was 90-95%. For transduction of quiescent HSCs, CD34⁺ cells were maintained in IMDM medium containing 20% BIT 9500 and 1 mM Pen/Strep. For pre-stimulation, CD34⁺ cells were maintained in the above medium supplemented with 50 ng/ml each of TPO, G-CSF, and IL-6, 100 ng/ml of Flt-3, and 150 ng/ml of SCF for 24 h. Transduction was carried out in the respective medium for 12 h in the presence of 4 μg/ml polybrene, on 1-2.5E4 cells seeded per well in round-bottom 96-well plates in a total volume of 150 ul. Following transduction, the medium was replaced with IMDM supplemented with 10% FBS, 1 mM Pen/Strep, 50 ng/ml each of IL-3 and IL-6, and 100 ng/ml SCF for in vitro expansion. Transduced cells were cultured for 11-14 days with medium change every 2-3 days and splitting as necessary. GFP expression was assessed by flow cytometry using a BD FACSCalibur.

Colony forming assay, NSG mouse reconstitution, and serial transplantation. For colony forming assays, transduced cord blood CD34⁺ HSPCs were counted and 100 cells were seeded in 1.5 ml methocult4434 (StemCell Technologies) in 30 mm dishes in triplicate. Total BFU-E, CFU-GM, CFU-M, and CFU-GEMM colonies, as well as GFP+ colonies, were counted 16-18 days after plating using a fluorescent microscope. For NSG reconstitution, 8-10 week-old mice were irradiated with 230 cGy using a cesium source, and injected retro-orbitally with transduced and washed cord blood CD34⁺ cells (2-3E5 cells/recipient) within 24 h of irradiation. Mice were bled every 4 weeks starting from 8 weeks post injection, and sacrificed at 19 weeks to assess engraftment and GFP expression in the bone marrow, spleen, and thymus by flow cytometry. For serial transplantation, primary recipients were sacrificed at 12 weeks post injection, and bone marrow cells from both femurs of one mouse were injected into two irradiated secondary recipients, which were again sacrificed at 12 weeks post injection for flow cytometric analysis of bone marrow and spleen. NSG mice were maintained at the Scripps Research Institute Molecular and Experimental Medicine animal facility.

Virion entry assay. Stimulated cord blood CD34⁺ cells (3.5E5) were transduced with BLAM-Vpr containing vectors at an MOI of 25 in the presence of 20 μg/ml rapamycin or DMSO. After the 12-hour transduction, cells were washed and resuspended in 250 ul loading medium containing 20% BIT9500 in IMDM without antibiotics. The assay was carried out according to manufacturer's instructions (Invitrogen LiveBlazer FRET B/G loading kit with CCF4-AM). Briefly, 50 ul 6x substrate loading was added to the cell suspension in a 24-well plate to the final concentration of 1E6 cells/ml. The reaction was allowed to develop in the dark at room temperature for 7-8 hours. Cells were then washed twice, and fixed in FACS buffer containing 1% PFA. The proportion of cells exhibiting blue and green fluorescence were read on a BD LSRII equipped with a UV laser in the pacific blue and amCyan channels, respectively. The amount of viral entry was determined by the ratio of blue-to-green fluorescence.

Quantification of HIV-1 reverse transcription products. Stimulated cord blood CD34⁺ cells (2-3E5) were transduced with an MOI of 25 in the presence of 20 μg/ml rapamycin or DMSO, and harvested at 6, 12, or 24 hours after the start of transduction by freezing cell pellets at −80° C. Total DNA was extracted using the QiaAmp DNA mini kit, and treated with Dpnl for 2-4 hours to eliminate plasmid DNA. Quantitative PCR was carried out on the Roche LightCycler 480 using previously published primer and probe sequences (Prasad et al., HIV Protocols: Second Edition 485, 2009).

Cell cycle assay. Stimulated cord blood CD34⁺ cells (6E5 cells) were treated with DMSO or 20 μg/ml rapamycin for 6 hours. Following treatment, cells were washed twice, resuspended in 10 μl PBS, and fixed by adding 100 μl 70% ice-cold ethanol and immediately vortexing. Fixation was completed by storing the cells at −20° C. for at least 24 hours. To determine cell cycle distribution by DNA content, fixed cells were washed, treated with 1 μg/μl RNase A (Invitrogen) at 37° C. for 30 minutes, resuspended in FACS buffer containing 25 μg/ml propidium iodide (Invitrogen), and characterized on a BD FACSCalibur by fluorescence in the FL3 channel.

p21 mRNA quantification. Stimulated cord blood CD34⁺ cells (1.6E5) were treated with 20 μg/mIrapamycin or DMSO for 6 hours, and harvested by flash freezing cell pellets in liquid nitrogen to preserve RNA. Total RNA was isolated using the Qiagen RNeasy Plus mini kit, treated with DNase I, and reverse transcribed using SuperScript II RT with oligo d(T) primers (Invitrogen). Expression of the p21 gene CDKN I A was quantified using a Taqman gene expression assay (Applied Biosystems #4453320). Expression of the reference gene GAPDH was quantified using SYBR Green chemistry and the following primers at 500 nM: forward AGCAATGCCTCCTGCACCACCAAC (SEQ ID NO:1); reverse CCGGAGGGGCCATCCACAGTCT (SEQ ID NO:2). Quantitative PCR reactions were run on the Roche LightCycler 480.

### Example 2 Rapamycin Increases Lentiviral Transduction Efficiency in Human CD34⁺ HSCs

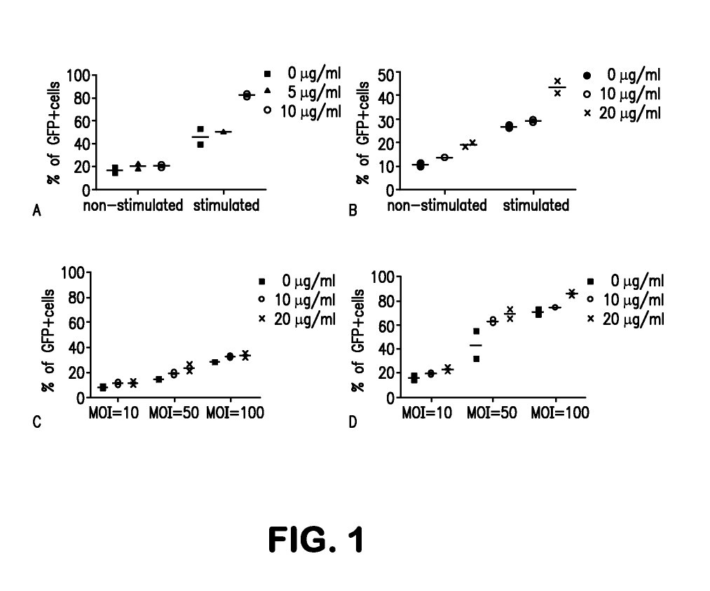

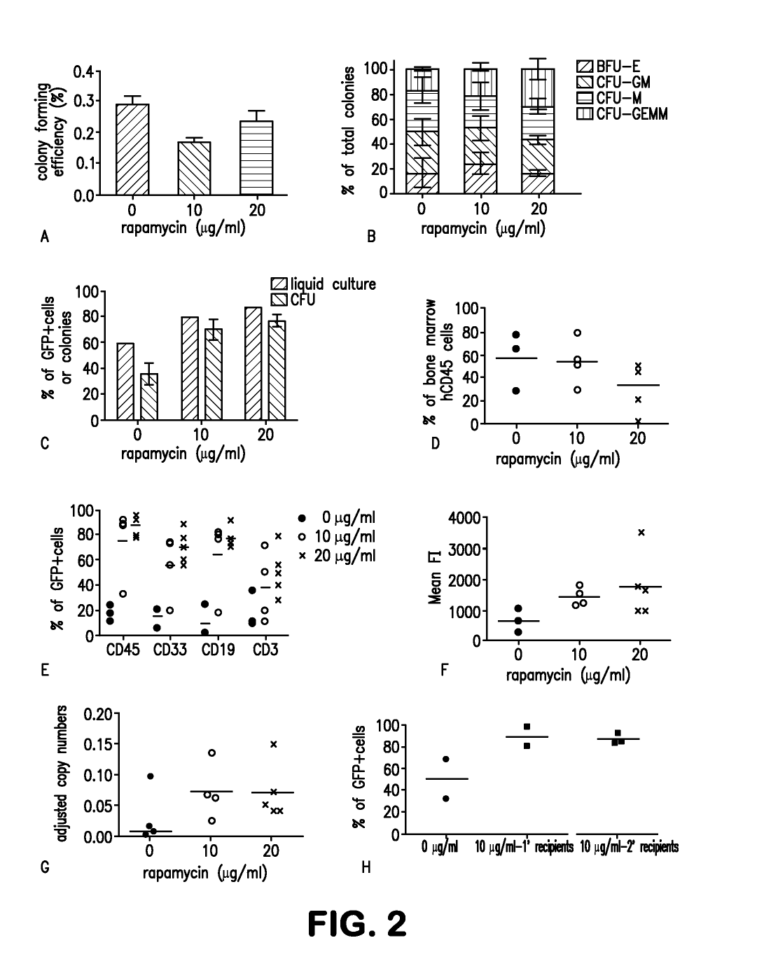

To determine whether rapamycin affects transduction efficiency of human HSCs by HIV-1 based lentiviral vectors, we transduced CD34⁺ cells isolated from human cord blood (purity>90%), with or without cytokine pre-stimulation, in the presence of various concentrations of rapamycin. Presence of rapamycin during transduction resulted in a general increase in transduction efficiency, as indicated by the percentage of GFP-expressing cells after 11-14 days in culture (FIG. 1A). The magnitude of increase was affected by the cytokine environment; the effect was minor in non-stimulated cells, and more pronounced in pre-stimulated cells, with a two-fold increase from 40% to 80% GFP positivity. We further tested a range of multiplicities of infection (MOI) in non-stimulated (FIG. 1C) and stimulated cord blood CD34⁺ cells (FIG. 1D) and observed rapamycin-induced transduction increase at each MOI. Out of the three MOIs tested, the greatest effect was seen at an MOI of 50, likely because a critical level of vector input had not been reached at MOI of 10, while baseline transduction was too high to show further increase at MOI of 100. We also tested the effect of rapamycin on the transduction of adult bone marrow CD34⁺ cells, which are a relevant cell source for adult HSC gene therapy. We found a two-fold increase in transduction efficiency under both non-stimulated and stimulated conditions, confirming that rapamycin facilitates transduction of CD34⁺ cells from both adult and neonatal origins (FIG. 1B). Since cord blood cells showed more pronounced increase in transduction efficiency following pre-stimulation, we carried out subsequent experiments in pre-stimulated cells.

### Example 3 Rapamycin Increases Lentiviral Transduction Efficiency in NOD/SCID/IL2y^{-/-} (NSG) Long-Term and Serial Repopulating Cells.

To determine whether primitive HSCs in the heterogeneous CD34⁺ population are transduced in the presence of rapamycin, we tested the ability of transduced cord blood CD34⁺ cells to engraft irradiated NSG mice. A fraction of transduced cells was assessed in parallel liquid culture and colony forming assays. Rapamycin did not significantly affect the efficiency or proportion of colony formation (FIG. 2A-B), while GFP expression was enhanced (FIG. 2C). Mice were sacrificed 19 weeks after injection to assess long-term engraftment. Reconstitution levels were not statistically different among control and rapamycin treatment groups, indicating that rapamycin did not impair long-term engraftment ability of CD34⁺ cells (FIG. 2D). GFP expression in human CD45⁺ cells in mouse bone marrow was significantly increased in a dose-dependent manner, with 20 μg/ml rapamycin-treated group showing a four-fold enhancement over the control group (80% vs 20% GFP positivity) (FIG. 2E). This four-fold transduction enhancement in NSG-repopulating cells was more pronounced than in parallel liquid culture or CFU assay (FIG. 2C), indicating preferential transduction of primitive HSCs in the presence of rapamycin. Increased GFP expression was observed across all myeloid and lymphoid lineages, further confirming the transduction of multipotent HSCs (FIG. 2E). Mean fluorescent intensity was also increased following rapamycin treatment, but to a lesser degree than the percentage of GFP-expressing cells, with the only statistically significant difference between 0 and 10 μg/ml rapamycin groups (FIG. 2F). This is mirrored by non-significant increases in inserted provirus copy numbers (FIG. 2G).

To stringently establish transduced cells as primitive HSCs, we carried out serial NSG mouse transplantation with cord blood CD34⁺ cells transduced with or without rapamycin. Primary recipient mice were sacrificed 12 weeks post injection, and bone marrow cells were characterized and injected into secondary recipients. Bone marrow cells of secondary recipients were again analyzed 12 weeks post injection. We found transplantable human cells that maintained high levels of GFP positivity in the secondary recipients, further confirming that primitive HSCs were transduced to high levels in the presence of rapamycin (FIG. 2H).

### Example 4 Rapamycin Increases Transduction Efficiency of Integrase-Defective Lentiviral Vectors (IDLVs)

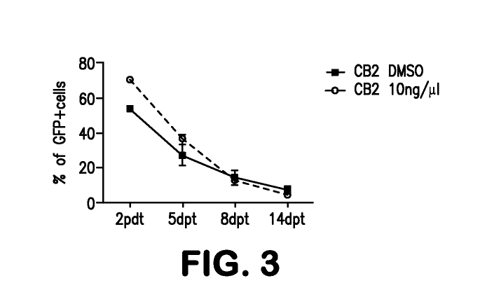

IDLVs are advantageous over conventional integrating lentiviral vectors for transient gene expression in dividing cells or stable expression in terminally-differentiated cells, as they eliminate the risk of insertional mutagenesis. We investigated whether IDLY transduction of HSCs benefit from rapamycin treatment. Cord blood CD34⁺ cells were transduced with IDLVs in the absence or presence of rapamycin, and GFP expression was followed from 2-14 days post transduction. Rapamycin treatment increased the percentage of GFP expressing cells by 17% two days after transduction, a difference that gradually dissipated within two weeks (FIG. 3). This shows that rapamycin can enhance transduction by IDLVs, as genomic integration is not a prerequisite.

### Example 5 Rapamycin Increases Transduction Efficiency of Wild Type and Integrase-Defective Lentiviral Vectors in Mouse Lin− Cells

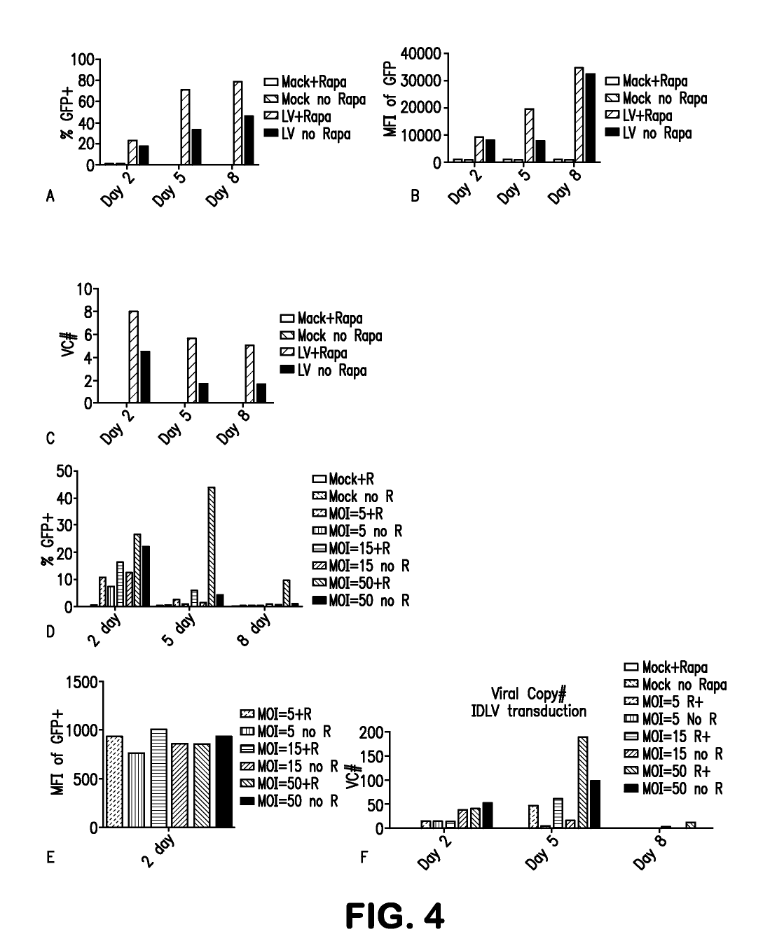

Mouse hematopoietic development is extensively characterized and is the model system of choice for human hematopoiesis. We therefore examined whether rapamycin enhances lentiviral transduction of mouse Lin− HSCs. We transduced mouse Lin− cells in the presence of rapamycin, and found increased percentages of GFP expression, mean fluorescent intensities, and provirus copy numbers using either integrating lentiviral vectors (FIG. 4 A-C) or IDLVs (FIG. 4D-F). Therefore, rapamycin increases lentiviral transduction efficiency in mouse Lin− cells, similar to human CD34⁺ cells.

### Example 6 The Effect of Rapamycin is Specific to HSCs and not Observed in Differentiated Myeloid or T cells

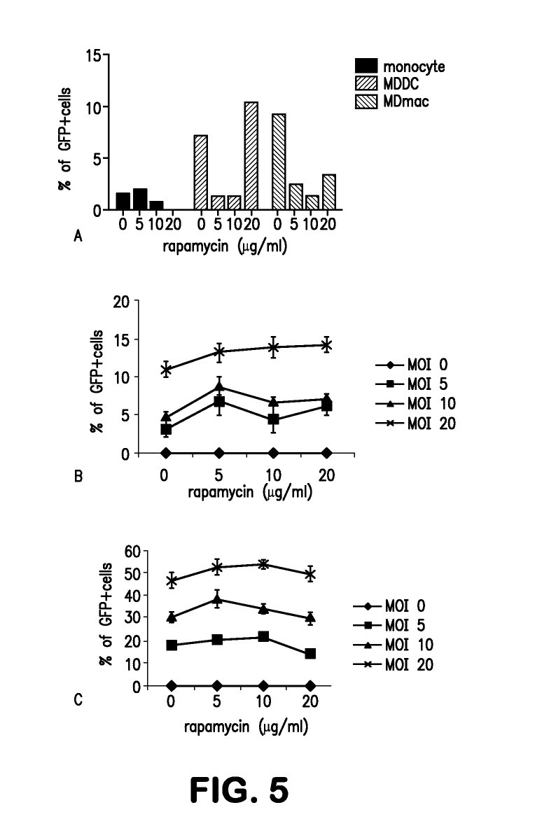

We asked whether susceptibility to rapamycin-mediated transduction enhancement is preserved throughout hematopoietic development. We transduced primary human monocytes, monocyte-derived dendritic cells and macrophages, and CD4⁺ T cells in the absence or presence of rapamycin. Unlike in HSCs, transduction efficiency was decreased by rapamycin treatment in primary myeloid cells (FIG. 5A), and unchanged in resting and activated CD4⁺ T cells (FIG. 5B-C). Thus, rapamycin-mediated transduction enhancement appears to be a phenomenon specific to primitive hematopoietic cells.

Example 7 The effect of rapamycin is indirect and is required early in transduction

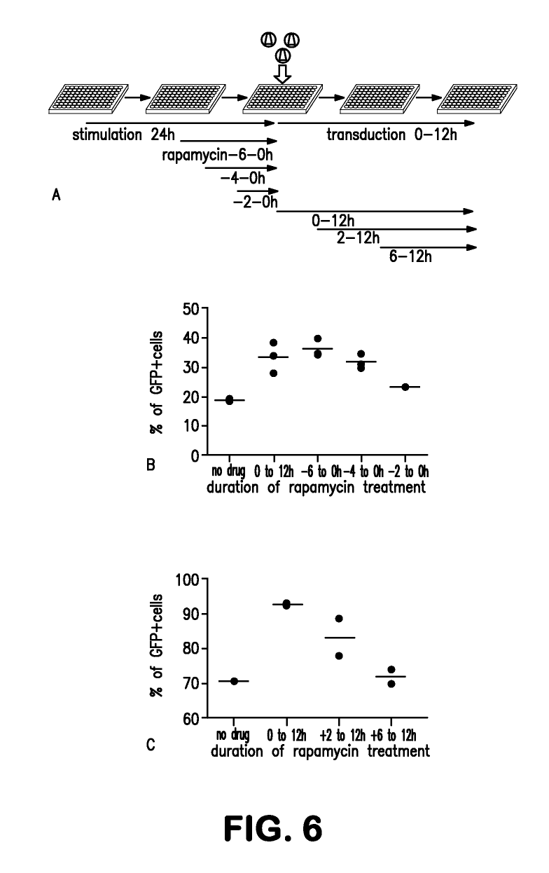

We investigated the temporal aspect of rapamycin-mediated transduction enhancement by varying the timing and duration of rapamycin treatment (schematized in FIG. 6A). We pre-treated cord blood CD34⁺ cells with rapamycin for 2-6 hours, then transduced in the absence of rapamycin. 4-6 hours of pre-treatment resulted in equivalent transduction enhancement as 12 hours of treatment concurrent with transduction (FIG. 6B), indicating that rapamycin does not act directly on incoming vectors but rather induces cellular states more amenable to transduction. We then delayed the addition of rapamycin by 0-6 hours after vector addition. Transduction enhancement was abolished by a 6-hour delay in rapamycin addition (FIG. 6C), indicating that the action of rapamycin is required early in transduction.

### Example 8 Rapamycin Enhances Lentiviral Entry and Reverse Transcription

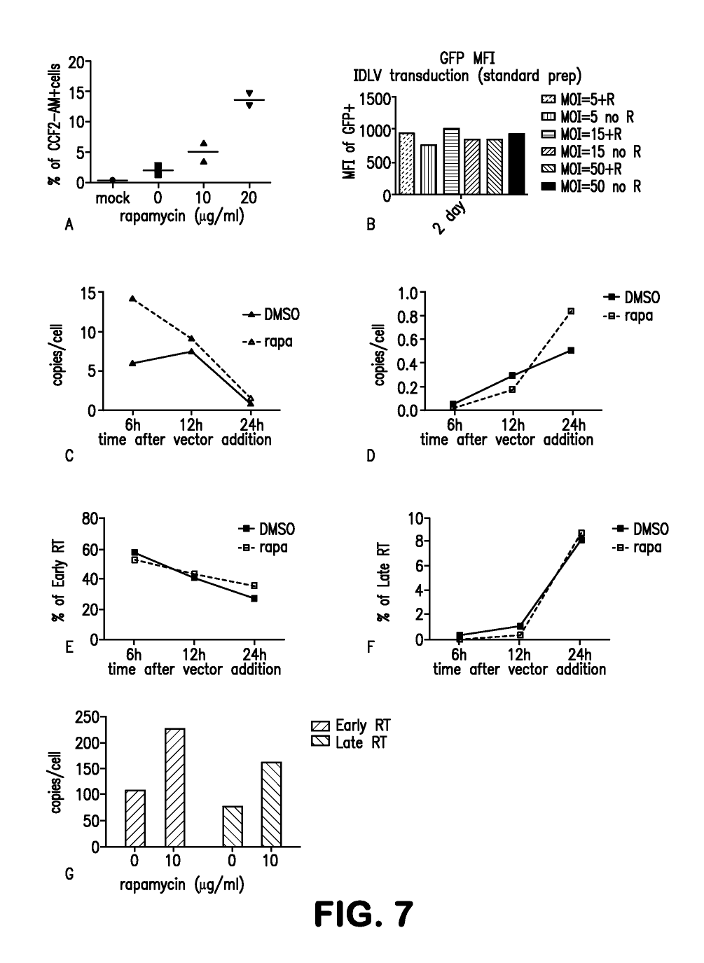

We examined the kinetics of vector entry and reverse transcription to elucidate the replication step facilitated by rapamycin. The amount of cytoplasmic entry of viral cores can be determined by transducing cells with a vector carrying the HIV-1 accessory protein Vpr fused to (3-lactamase (BLAM), and quantifying the blue fluorescence of a cleaved BLAM substrate by flow cytometry (see, e.g., Tobiume et al., J. Virol. 77:10645-50, 2003). We found dose-dependent increase in vector entry into the cytoplasm of cord blood CD34+ cells following rapamycin treatment (FIG. 7A). In addition, we quantified the amount of reverse transcription and nuclear import products by qPCR, and found 2-to-3-fold increase in the per cell amount of viral strong-stop DNA, full-length DNA, and 2-LTR circles in the presence of rapamycin (FIG. 7B-D). Therefore, rapamycin appears to act by increasing cytosolic delivery of viral cores, providing more templates for subsequent reverse transcription and nuclear import. The ratios of viral DNA species between each pair of adjacent steps were similar with or without rapamycin treatment, indicating that the efficiencies of reverse transcription and nuclear import were unaffected (FIG. 7E-F). IDLV transduction also resulted in two-fold increases in strong-stop and full-length viral DNA in the presence of rapamycin, consistent with wild type vector (FIG. 7G).

### Example 9Maximal Transduction Efficiency Requires Autophagy but not Accumulation of Autophagosomes

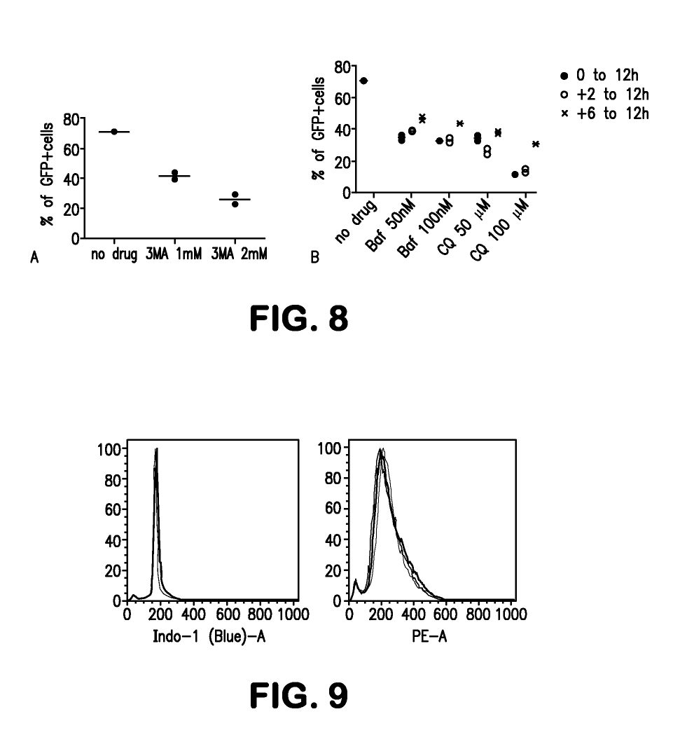

To determine whether autophagy induction is responsible for rapamycin-mediated transduction enhancement, we transduced cord blood CD34⁺ cells in the presence of compounds that modulate various steps of autophagy. Inhibiting autophagosome formation with 3-methyadenine decreased GFP expression, highlighting a requirement for basal autophagy (FIG. 8A). Since non-degradative stages of autophagosome formation may promote HIV-1 replication, we used bafilomycin A1 and chloroquine to inhibit fusion with lysosomes, which leads to an accumulation of non-acidified autophagosomes. Transduction efficiency was decreased, even when addition of the molecules was delayed to allow for endocytic entry of VSV-G pseudotyped vectors (FIG. 8B). Therefore, the effect of rapamycin was not due to an accumulation of autophagosomes.

### Example 10 Rapamycin does not Affect Cell Cycle Distribution of Cord Blood CD34⁺ Cells

Alternative non-autophagic mechanisms could potentially account for transduction enhancement by rapamycin. Rapamycin induces cell cycle arrest at the GI phase by blocking GUS progression in some cell types. A change in cycling status, specifically accumulation in GI phase, increases the permissivity of HSCs to lentiviral transduction. We therefore characterized cell cycle distribution of CD34⁺ cells by DNA and RNA content, and found no change between control and rapamycin-treated cells (FIG. 9). Therefore, rapamycin-mediated transduction enhancement is not due to cell cycle modulation.

### Example 11 Rapamycin does not Down-Regulate p21/Cip1/Waf1

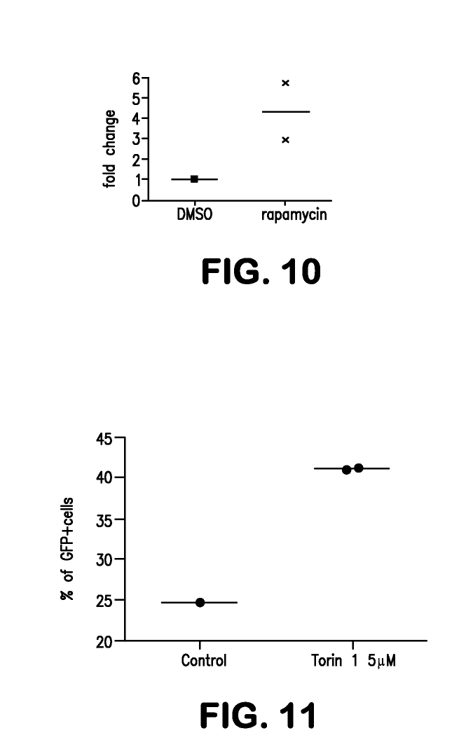

The CDK inhibitor p21/Cip1/Waf1 has recently been identified as a novel HIV-1 restriction factor in hematopoietic cells. It is up-regulated at both mRNA and protein levels in CD4⁺ T cells of HIV-1 elite controllers, and is associated with impairment in HIV-1 reverse transcription. In HSCs p21 has been shown to restrict both HIV-1 and lentiviral vectors at the level of integration. Rapamycin is reported to modulate p21 expression in a variety of cell types. We therefore speculated that rapamycin may reduce p21 levels in HSCs, thereby relieving anti-HIV-1 restriction. However, we found 3-5-fold up-regulation in p21 mRNA in rapamycin-treated cord blood CD34⁺ cells by RT-PCR, contradicting a role for p21 in restriction that is overcome by rapamycin (FIG. 10).

Example 12 ATP-Competitive Inhibitor Torin 1 Enhances Viral Transduction Efficiency

Rapamycin inhibits mTOR kinase activity of mTOR complex 1 (mTORC1) in an allosteric manner by recruiting the cytoplasmic protein FKBP12. However, rapamycin-FKBP12 cannot interact with mTOR complex 2 (mTORC2), which carries out functions both redundant and distinct from mTORC1. Torin 1 is a member of a new class of ATP-competitive active site inhibitors of mTOR, and is thus able to inhibit both mTOR-containing complexes mTORC1 and mTORC2.

To assess potential contribution of mTORC2 to transduction enhancement, we transduced human cord blood CD34⁺ cells in the presence of 5 μM of Torin 1. The results as shown in FIG. 11 indicate that Torin 1 enhanced in vitro transduction efficiency relative to DMSO control. The enhancement is comparable to the effect observed with rapamycin.

### Example 13 mTOR Inhibition Enhances Transduction Via Multiple Endocytic Entry Mechanisms with Distinct Receptor Usage

To investigate whether the transduction enhancing effect of mTOR inhibitors (e.g., rapamycin) is specific to VSVG-pseudotyped vectors, we tested lentiviral vectors pseudotyped with the Lassa virus glycoprotein (LASV). LASV and the closely related lymphocytic chriomeningitis virus glycoprotein (LCMV) are alternative envelopes being explored for gene therapy, and have lower cellular toxicity compared to traditional VSVG envelope. Like VSVG, LASV mediates pH-dependent viral entry through the endocytic pathway; but unlike VSVG, LASV entry is independent of classical endocytic components including clathrin, caveolin, dynamin, or actin.

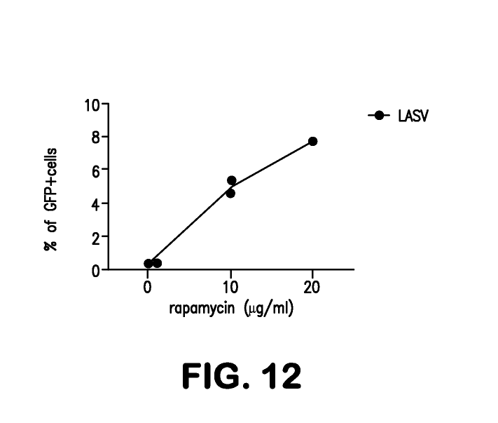

As shown in FIG. 12, we found that the efficiency of LASV-pseudotyped vector transduction of human CD34⁺ cells, while much lower than that of VSVG, was markedly enhanced by rapamycin treatment. Therefore, mTOR inhibition, e.g., via rapamycin, can facilitate multiple endocytic entry mechanisms with distinct receptor usage.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

1. A method for enhancing transduction efficiency of a viral vector into a stem cell, comprising transducing the stem cell with the vector in the presence of a compound that inhibits or antagonizes mTOR Complex 1 (mTORC1) and/or mTOR Complex 2 (mTORC2).

2. The method of claim 1, wherein the compound is an mTOR inhibitor.

3. The method of claim 2, wherein the mTOR inhibitor is rapamycin or analog compound thereof.

4. The method of claim 2, wherein the mTOR inhibitor is an ATP-competitive inhibitor.

5. The method of claim 1, wherein the viral vector is a recombinant retroviral vector, an adenoviral vector or an adeno-associated viral vector.

6. The method of claim 1, wherein the viral vector is a lentiviral vector.

7. The method of claim 1, wherein the viral vector is a HIV-1 vector.

8. The method of claim 1, wherein the stem cell is a hematopoietic stem cell (HSC), an embryonic stem cell or a mesenchymal stem cell.

9. The method of claim 1, wherein the stem cell is a hematopoietic stem cell.

10. The method of claim 1, wherein the stem cell is isolated from umbilical cord blood, peripheral blood or bone marrow.

11. The method of claim 1, wherein the stem cell is human CD34⁺ cell.

12. The method of claim 1, wherein the stem cell is pre-stimulated with at least one cytokine prior to transduction of the vector.

13. The method of claim 12, wherein the at least one cytokine is TPO, CSF, IL-6, Flt-3 or SCF.

14. The method of claim 1, wherein the vector is transduced into the stem cell at a multiplicity of infection (MOI) of 5, 10, 25, 50 or 100.

15. The method of claim 1, wherein the compound is present during the entire transduction process or at specific intervals.

16. The method of claim 1, wherein the viral vector encodes a therapeutic agent.

17. The method of claim 1, wherein the viral vector is a non-integrating lentiviral vector.

18. A kit for delivering a therapeutic agent into a target cell with enhanced targeting frequency and payload delivery, comprising (a) a viral vector encoding the therapeutic agent, and (b) an inhibitor of mTOR complexes.

19. The kit of claim 18, wherein the inhibitor is an mTOR inhibitor.

20. The kit of claim 19, wherein the mTOR inhibitor is rapamycin or an analog thereof.

21-27. (canceled)
